(19) United States
(12) Patent Application Publication
(10) Pub. No.: US 2014/0272762 A1
(43) Pub. Date: Sep. 18, 2014

(54) APPARATUS AND METHOD FOR DETERMINING TOOTH SHADE

(71) Applicant: BRUCE HENRY PROPERTIES, LLC DBA BONADENT, Seneca Falls, NY (US)

(72) Inventor: Joseph Arnold Weisz, Seneca Falls, NY (US)

(73) Assignee: Bruce Henry Properties, LLC dba BonaDent, Seneca Falls, NY (US)

(21) Appl. No.: 13/836,348

(22) Filed: Mar. 15, 2013

(51) Int. Cl. A61C 19/10; A61B 5/00

(52) U.S. Cl. CPC A61C 19/10; A61B 5/0088
USPC 433/26; 433/203.1

(57) ABSTRACT

A shade matching device for at least one artificial tooth or crown restoration is disclosed comprising eight shade guides held by three gingival colored holders. The shade guides are grouped by value and arranged within a particular value by chroma.

A method for determining natural tooth shade is also disclosed. Images of the target tooth are captured with the shade guides placed adjacent to the target tooth. Preferably, additional images are captured to provide additional data, including images when the patient's lips are formed in a smile; in the M position; and retracted. A close-up image of the patient's teeth is also captured along with an image that includes the patient's full face.

This disclosure relates to an apparatus for determining tooth shade and other tooth characteristics for manufacturing artificial teeth and/or crowns. This disclosure also relates to a method for determining tooth shade and other tooth characteristics for purposes of communicating the proper shade and other necessary information to the dental laboratory for use in manufacturing artificial teeth and/or crowns, or other dental devices.

### BACKGROUND OF THE INVENTION

Selecting the shade for an artificial tooth or crown is one of the more difficult aspects of restorative dentistry. In providing dental treatment for both medical and cosmetic purposes, a goal is to create a prosthetic (such as a tooth or crown restoration) with the same color as the adjacent natural teeth. Manufacturing such prosthetics therefore generally requires a precise discrimination of the color of the tooth so that the prosthetic can be manufactured precisely to the natural tooth color. Natural teeth have a wide variation in value and hue. Determining the correct match to existing natural teeth to create artificial teeth and/or crowns is difficult and prone to error due to the subjectivity of the determination. In addition, contour and other factors, such as degree of fluorescence, opalescence, and enamel conditions (including thickness, texture and/or luster) influence the apparent value and hue of the teeth.

The shade of natural teeth is measured by hue, chroma and value. “Hue” is the factor that distinguishes one family of colors from another. See James Fondriest, Shade Matching in Restorative Dentistry: The Science and Strategy, 23(5) Int. J. Periodontics Restorative Dent. 467, 467 (2003). Generally, natural teeth tend to have either a reddish or yellowish hue. “Chroma” is the intensity or saturation of the color. In general, increased chroma makes the shade appear darker, so that the “value” decreases correspondingly. “Value” is the brightness of the color, or the amount of light that is returned from an object. See Fondriest, supra, at 468. The most important characteristic in determining the shade of a tooth for the purposes of determining the proper shade for a prosthetic is the value. The chroma and hue of the natural tooth are less important than the value. Additionally, the contour and shape of the teeth are more significant than the hue. Tooth contour and shape influence the hue, chroma and value of the teeth by varying how light interacts with the tooth surface.

For some time, shade guides have been used as color samples for determining the color of natural teeth. A shade guide contains a plurality of shade tabs arranged by some variable (traditionally by hue). The shade tabs are generally formed from plastic and shaped like a tooth. Each shade tab has a specific hue, chroma and/or value. Shade tabs have been traditionally mounted on a stick for ease of handling. The shade tabs generally indicate value and chroma by a numerical value placed on the stick and hue by a letter placed on the stick. The dentist places one or more shade tabs next to the patient's tooth for which a color match is desired and compares the shade of the natural tooth to the shade of the shade tab. The dentist will, based on observation, determine which of the shade tabs has the shade most closely corresponding to that of the patient's tooth. The dentist will then provide the value, hue and chroma values on a prescription to the prosthetic manufacturer. In addition to providing the base value designating the particular value, hue and chroma desired for the prosthetic, the prescription will provide additional information about the desired shade, such as requests to add a particular hue to the base shade at a particular point on the prosthetic; to add calcification to the tips; and/or to slightly alter the value or hue. The extra comments or requests in the prescription were traditionally the only method to address additional factors affecting the tooth shade such as opacity, translucency and transparency.

In each case, determining the shade of the tooth to be matched depends on the dentist's subjective evaluation of the shade. Shade determinations vary by individual to individual and often upon the environmental conditions in which the observations are made. Determinations of shade, therefore, are highly variable and tend to be unreliable. Manufacturing a prosthetic where the shade does not match the shade of the selected natural tooth results in additional costs due to the need to alter the prosthetic or manufacture a new prosthetic, and in lost time due to the need to engage in additional shade selection and fitting of the prosthetic.

Traditional shade guides separated tooth color into four separate hues: A—Brown; B—Yellow; C—Gray and D—Red. Such guides represented chroma, or color intensity, by numbers ranging from 1 through 4. Early shade guides arranged the shade tabs (16 in number) first by hue and then, within each due, by chroma. It was difficult to obtain an accurate assessment of tooth shade using those guides because the most important factor in tooth shade, value, was not measured. In addition, environmental factors such as lighting and the positioning of the patient caused inaccuracies in determining the shade.

Certain shade guides include value as a variable, grouping the shade tabs by value (ranging from 1 to 5). Within certain of the value groupings (2, 3 and 4), the shades are separated into hue by three different values: L (light), indicating a yellow hue; M (medium), indicating a yellow-red or orange hue; and R (red), indicating a red hue. In addition, within each value grouping (1 through 5), shade tabs are arranged with the chroma increasing from top to bottom. These shade guides contain 29 tabs, representing tooth shades with a variety of values, hues and chromas. The dentist or assistant determines the tooth shade by first determining the value of the patient's natural tooth by holding up the different value groups to the tooth. The hue is then determined by holding up the different hue tabs (L, M or R) within the value group to the tooth, and then finally by determining the chroma. As with earlier shade guides, the prescription is conveyed to the manufacturer by an alphanumerical designation indicating the base shade (for example, 3M2) along with any request for alterations in the hue or chroma and specifics about tooth characteristics such as contour and surface texture. Again, this process requires the subjective evaluation of the dentist or assistant and is prone to inaccuracies. Further, the number of tabs in the shade guide causes confusion and making the various matches is time-consuming. Even with attempts to rearrange the shade tabs in a linear matter and combining the chroma/hue values, problems of complexity and inaccuracy remain.

Other shade guides have attempted to increase the accuracy of determining tooth shade. For example, Yamamoto, U.S. Pat. No. 5,989,022 disclosed a gingival colored holder with concaves shaped to fit the neck part of the shade tab. This holder sought to correct the color mismatches that resulted from using the traditional sticks to hold the shade tab, which caused a contrast effect that altered the perceived color of the shade tab when compared to the natural tooth, which was in contact with the gingiva. Other types of shade arrangements have been proposed, for example in Yamamoto, U.S. Pat. Pub. No. 2010/0173257 A1. Traditional shade tab holders, however, do not have a sufficiently close fit to the shade tab and also do not display the variation of natural tissue.

Current shade guides also have several limitations in that they cannot account for all of the variability found in natural teeth. See Irfan Ahmad, PROTOCOLS FOR PREDICTABLE AESTHETIC DENTAL RESTORATIONS 93 (Blackwell Munksgaard 2006). Factors that current shade guides do not capture include the degree of fluorescence, the degree of enamel translucency, enamel texture, and the degree of opalescence. For example, translucency has been defined as the gradient between transparent and opaque. Even if the hue, chroma and value are the same for two teeth, different translucency gradients will cause a different appearance due to the scattering of light within the porcelain, which occurs with increased translucency. See Fondriest, supra. Likewise, differences in fluorescence and opalescence can cause teeth with the same hue, chroma and value to appear different. Measuring these factors is an inherently subjective process, complicated by the lack of effective means of communicating the data to the manufacturer. Further, determining the desired contour and surface characteristics for the prosthetic is also subjective, and the same difficulties in communicating the data to the manufacturer exist.

Further, determination of tooth shade is complicated by variables that affect shade taking. Similar shades will appear different depending on the conditions existing during the observation, including the light source type and angulation, size and shape differences in the tooth, background color (from sources such as lipstick, bright clothing or the wall) and perception angle (eye level as compared to the location of the teeth). While various methods, including certain electronic measurement devices, have been developed to reduce subjectivity and variability in shade determination, they have not been entirely successful. Even using current digital means of shade determination, where an image is taken of both the shade tab and the target tooth, and software is used to compare the CIELab color coordinates of both the tab and the tooth, significant inaccuracies still result, leading to the need to retouch or refabricate prosthetics.

### BRIEF SUMMARY OF THE INVENTION

Disclosed is a shade matching device for at least one artificial tooth or crown restoration, which comprises eight shade guides and three gingival colored holders in which the shade guides are located. The shade guides are arranged in the three gingival colored holders grouped by value and arranged within a particular value by chroma. In a preferred embodiment of the device, the two shade guides having the highest value are located in the first gingival colored holder, arranged by chroma; the three shade guides having a medium value are located in the second gingival colored holder, arranged by chroma; and the three shade guides having the lowest value are located in the third gingival colored holder, arranged by chroma.

Another preferred embodiment of the device comprises eight shade guides, made with an acrylic material in the shape of a tooth with a neck portion. The device also comprises three gingival colored holders, each of which has at least two concave shapes corresponding to the neck shape of the portion of the artificial tooth. The shade guides are mounted in the holder such that the labial portion of the neck is fitted against the gingival colored holder. The shade guides are arranged in the three gingival colored holders grouped by value and arranged within a particular value by chroma. Preferably, the two shade guides having the highest or brightest value are located in the first gingival colored holder, arranged by chroma. The three shade guides having a medium value are located in the second gingival colored holder, arranged by chroma. The three shade guides having the lowest or darkest value are located in the third gingival colored holder, arranged by chroma.

Also disclosed is a method for determining natural tooth shade for purposes of having prosthetic teeth or crowns manufactured. In accordance with the disclosed method, the patient is seated in an upright position and a camera is placed perpendicular to the facial surfaces of the patient's teeth. An image of the target tooth is captured with a digital image capture means where a shade guide of the shade matching device is placed adjacent to said target tooth. Additional images of the target tooth are captured with the digital image capture means, with an image being captured for each of the remaining shade guides of the shade matching device and where the shade guides are each placed adjacent to said target tooth. Any embodiment of the disclosed shade matching device may be used in performing the steps of the disclosed method. The steps of the disclosed method may be performed in any order.

Preferably, additional images are captured to provide additional data for determining the contour and texture of the natural tooth. An image of the target tooth is captured with a digital image capture means when the patient's lips are formed in a smile; when the patient's lips are in the M position; and when the patient's lips are retracted. In addition, an image is captured that is a close-up of the patient's teeth showing four to six maxillary and four to six mandibular anterior teeth. Finally, an image of the target tooth is captured with a digital image capture means where the image includes the patient's full face.

The digital images are transmitted to the dental laboratory or other prosthetic manufacturer and converted to gray scale to determine the correct value of the natural tooth. The digital images are also used to determine the hue and chroma of the target tooth, as well as its contour and texture. The method facilitates not only the transmission of data sufficient to determine the hue, value and chroma of the target tooth accurately but also facilitates the transmission of information regarding micro-esthetics (texture and characterization), gingival contour and occlusion, the visibility of the teeth under normal conditions, and other information vital to matching the target tooth.

### BRIEF DESCRIPTION OF THE DRAWING

FIG. 1 is the depiction of a preferred embodiment of the disclosed device showing the composition and arrangement of the shade tabs.

### DETAILED DESCRIPTION OF THE INVENTION

FIG. 1 illustrates a preferred embodiment of a shade matching device 1 for artificial teeth comprised of three separate shade tab holders 2 in which at least eight separate shade tabs 3 are arranged. The shade tab holders 2 are gingival colored to reduce the contrast effect and are manufactured from acrylic or similar material to simulate natural gums. The holders have one or more concaves 4 in which the neck part of the shade tab is fitted when the shade tab is mounted in a holder 2. The first shade tab holder comprises two shade tabs with a bright value (1) and medium hue, arranged in increasing chroma (1 and 2), as shown by the designations 1M1 and 1M2. The second shade tab holder comprises three shade tabs with a medium value (2) and medium hue, again arranged in increasing chroma (1, 2 and 3), as shown by the designations 2M1, 2M2 and 2M3. The third shade tab holder comprises three shade tabs with a darker or lower value (3) and medium hue, again arranged in increasing chroma (1, 2 and 3), as shown by the designations 3M1, 3M2 and 3M3. Although the shade tabs shown in FIG. 1 have designations such as 1M1, 1M2 etc. placed on the tab, these designations have been provided for the sake of the description and are not depicted on the actual shade tabs.

In performing the steps of the disclosed method to determine tooth shade, the preferred apparatus to capture the digital images is a DSLR digital camera with a macro setting, but any device that can capture high resolution digital images may be used. Before proceeding, it should be confirmed that the patient has not bleached his/her teeth within the last two weeks and does not plan to bleach the teeth before the prosthetic is in place. The patient should sit in a chair and the camera lens should be placed perpendicular to the facial surfaces of the patient's teeth. The teeth should be hydrated. The dentist or other person performing the tooth matching process should select a target tooth to be matched. Ideally, the target tooth should be adjacent to the area where the prosthetic will be placed or implanted. The first shade tab should be held edge-to-edge to the target tooth, ensuring that the tab and the target tooth are on the same axial plane. The entire target tooth and the surrounding soft tissue area should be exposed. The shade tab should then be photographed while placed edge-to-edge to the target tooth. Each of the remaining shade tabs should be held edge-to-edge to the target tooth and photographed in the same manner.

Next, several images of the mouth should be taken. Ideally, the camera settings should include aperture priority, F32, spot metering, large resolution and manual focus. The first image is a close-up of the patient with a full smile. The image should be captured from just below the patient's nose to above the lower lip. The patient should laugh as the image is captured. The second image is a close-up of the patient with the lips in an “M position.” Again, the image should be captured from just below the patient's nose to above the lower lip. The patient should be asked to say “Emma” or another word that produces a similar sound. The image should be captured at the moment at which the patient relaxes the lips after the word “Emma” is spoken.

The third image is a close-up of the patient with retracted lips. Clear retractions should be used to completely remove the lips from the frame of the image. The image should be captured from just below the patient's nose to above the lower lip. The patient should bite down completely and all of the posterior teeth and as much gingiva as possible should be captured on the image. The fourth image is a close-up of the teeth. Again, clear retractions should be used to completely remove the lips from the frame. The image should capture the four to six maxillary and mandibular anterior teeth. The patient should bite down while the image is being taken. The emphasis should be on the surface texture and characterization. Finally, a full face image should be taken. The settings on the camera for this image should be aperture priority, F 16, matrix metering, large resolution and manual focus. The patient should be standing directly facing the camera. The image should capture from the top of the patient's head to just below the chin.

After the above-referenced images are captured, they should be transmitted as a group to the dental laboratory or other prosthetic manufacturer. There, the images will be converted to grayscale to determine the correct brightness value. The unconverted color digital photographs are used to identify the hue and chroma, and other features of the natural tooth.

What is claimed is:

1. A shade matching device for at least one artificial tooth or crown restoration, which comprises:
at least eight shade guides;
three gingival colored holders in which the shade guides are mounted;
wherein the shade guides are arranged in the three gingival colored holders grouped by value and arranged within a particular value by chroma.

2. The shade matching device according to claim 1,
wherein the two shade guides having the highest value are located in the first gingival colored holder, arranged by chroma;
wherein the three shade guides having the next highest value are located in the second gingival colored holder, arranged by chroma; and
wherein the three shade guides having the lowest value are located in the third gingival colored holder, arranged by chroma.

3. A shade matching device for at least one artificial tooth or crown restoration, which comprises:
eight shade guides, each formed in the shape of a tooth and having a neck portion;
three gingival colored holders, each of which has at least two concave shapes, said shapes corresponding to the neck shape of the portion of said artificial tooth, wherein the shade guides are mounted in said holder such that the labial portion of the neck is fitted against the gingival colored holder;
wherein the shade guides are arranged in the three gingival colored holders grouped by value and arranged within a particular value by chroma.

4. The shade matching device according to claim 3,
wherein the two shade guides having the highest value are located in the first gingival colored holder, arranged by chroma;
wherein the three shade guides having the next highest value are located in the second gingival colored holder, arranged by chroma; and
wherein the three shade guides having the lowest value are located in the third gingival colored holder, arranged by chroma.

5. A method of selecting the color of an evaluation target tooth with the use of a shade matching device comprised of multiple shade guides, the method comprising the steps of:
capturing an image of the target tooth with a digital image capture means where a shade guide of the shade matching device is placed adjacent to said target tooth; and
capturing additional images of the target tooth with a digital image capture means, with an image being captured for each of the remaining shade guides of the shade matching device and where the shade guides are each placed adjacent to said target tooth.

6. A method of selecting the color of an evaluation target tooth with the use of the shade matching device of claim 1, the method comprising the steps of:
capturing an image of the target tooth with a digital image capture means where a shade guide of the shade matching device is placed adjacent to said target tooth; and
capturing additional images of the target tooth with a digital image capture means, with an image being captured for each of the remaining shade guides of the shade matching device and where the shade guides are each placed adjacent to said target tooth.

7. A method of selecting the color of an evaluation target tooth with the use of the shade matching device of claim 2, the method comprising the steps of:
capturing an image of the target tooth with a digital image capture means where a shade guide of the shade matching device is placed adjacent to said target tooth; and
capturing additional images of the target tooth with a digital image capture means, with an image being captured for each of the remaining shade guides of the shade matching device and where the shade guides are each placed adjacent to said target tooth.

8. A method of selecting the color of an evaluation target tooth with the use of the shade matching device of claim 3, the method comprising the steps of:
capturing an image of the target tooth with a digital image capture means where a shade guide of the shade matching device is placed adjacent to said target tooth; and
capturing additional images of the target tooth with a digital image capture means, with an image being captured for each of the remaining shade guides of the shade matching device and where the shade guides are each placed adjacent to said target tooth.

9. A method of selecting the color of an evaluation target tooth with the use of the shade matching device of claim 4, the method comprising the steps of:
capturing an image of the target tooth with a digital image capture means where a shade guide of the shade matching device is placed adjacent to said target tooth; and
capturing additional images of the target tooth with a digital image capture means, with an image being captured for each of the remaining shade guides of the shade matching device and where the shade guides are each placed adjacent to said target tooth.

10. A method of determining the characteristics of a target tooth with the use of a shade matching device comprised of multiple shade guides, the method comprising the steps of:
capturing an image of the target tooth with a digital image capture means where a shade guide of the shade matching device is placed adjacent to said target tooth;
capturing additional images of the target tooth with a digital image capture means, with an image being captured for each of the remaining shade guides of the shade matching device and where the shade guides are each placed adjacent to said target tooth;
capturing an image of the target tooth with a digital image capture means when the patient's lips are formed in a smile;
capturing an image of the target tooth with a digital image capture means when the patient's lips are in the M position;
capturing an image of the target tooth with a digital image capture means when the patient's lips are retracted;
capturing an image of the target tooth with a digital image capture means where the imaged captured is a close-up of the patient's teeth showing four to six maxillary and four to six mandibular anterior teeth; and
capturing an image of the target tooth with a digital image capture means where the image includes the patient's full face.

11. A method of determining the characteristics of a target tooth with the use of the shade matching device of claim 1, the method comprising the steps of:
capturing an image of the target tooth with a digital image capture means where a shade guide of the shade matching device is placed adjacent to said target tooth;
capturing additional images of the target tooth with a digital image capture means, with an image being captured for each of the remaining shade guides of the shade matching device and where the shade guides are each placed adjacent to said target tooth;
capturing an image of the target tooth with a digital image capture means when the patient's lips are formed in a smile;
capturing an image of the target tooth with a digital image capture means when the patient's lips are in the M position;
capturing an image of the target tooth with a digital image capture means when the patient's lips are retracted;
capturing an image of the target tooth with a digital image capture means where the imaged captured is a close-up of the patient's teeth showing four to six maxillary and four to six mandibular anterior teeth; and
capturing an image of the target tooth with a digital image capture means where the image includes the patient's full face.

12. A method of determining the characteristics of a target tooth with the use of the shade matching device of claim 2, the method comprising the steps of:
capturing an image of the target tooth with a digital image capture means where a shade guide of the shade matching device is placed adjacent to said target tooth;
capturing additional images of the target tooth with a digital image capture means, with an image being captured for each of the remaining shade guides of the shade matching device and where the shade guides are each placed adjacent to said target tooth;
capturing an image of the target tooth with a digital image capture means when the patient's lips are formed in a smile;
capturing an image of the target tooth with a digital image capture means when the patient's lips are in the M position;
capturing an image of the target tooth with a digital image capture means when the patient's lips are retracted;
capturing an image of the target tooth with a digital image capture means where the imaged captured is a close-up of the patient's teeth showing four to six maxillary and four to six mandibular anterior teeth; and
capturing an image of the target tooth with a digital image capture means where the image includes the patient's full face.

13. A method of determining the characteristics of a target tooth with the use of the shade matching device of claim 3, the method comprising the steps of:
capturing an image of the target tooth with a digital image capture means where a shade guide of the shade matching device is placed adjacent to said target tooth;
capturing additional images of the target tooth with a digital image capture means, with an image being captured for each of the remaining shade guides of the shade matching device and where the shade guides are each placed adjacent to said target tooth;
capturing an image of the target tooth with a digital image capture means when the patient's lips are formed in a smile;
capturing an image of the target tooth with a digital image capture means when the patient's lips are in the M position;
capturing an image of the target tooth with a digital image capture means when the patient's lips are retracted;
capturing an image of the target tooth with a digital image capture means where the imaged captured is a close-up of the patient's teeth showing four to six maxillary and mandibular anterior teeth; and
capturing an image of the target tooth with a digital image capture means where the image includes the patient's full face.

14. A method of selecting the color of an evaluation target tooth with the use of the shade matching device of claim 4, the method comprising the steps of:
capturing an image of the target tooth with a digital image capture means where a shade guide of the shade matching device is placed adjacent to said target tooth;
capturing additional images of the target tooth with a digital image capture means, with an image being captured for each of the remaining shade guides of the shade matching device and where the shade guides are each placed adjacent to said target tooth; capturing an image of the target tooth with a digital image capture means when the patient's lips are formed in a smile;
capturing an image of the target tooth with a digital image capture means when the patient's lips are in the M position;
capturing an image of the target tooth with a digital image capture means when the patient's lips are retracted;
capturing an image of the target tooth with a digital image capture means where the imaged captured is a close-up of the patient's teeth showing four to six maxillary and four to six mandibular anterior teeth; and
capturing an image of the target tooth with a digital image capture means where the image includes the patient's full face.
